(19) United States
(12) Patent Application Publication
(10) Pub. No.: US 2014/0274783 A1
(43) Pub. Date: Sep. 18, 2014

(54) CHLAMYDIA-SPECIFIC CD8+ T CELLS AND METHODS OF ISOLATING

(71) Applicant: Raymond M. Johnson, Indianapolis, IN (US)

(72) Inventor: Raymond M. Johnson, Indianapolis, IN (US)

(21) Appl. No.: 14/215,144

(22) Filed: Mar. 17, 2014

Related U.S. Application Data
(60) Provisional application No. 61/787,048, filed on Mar. 15, 2013.

(51) Int. Cl. C12N 5/0783; G01N 33/569

(52) U.S. Cl. CPC C12N 5/0637; G01N 33/56977
USPC 506/9; 435/377; 506/12

(57) ABSTRACT

Isolated, Chlamydia-specific, IL-13 expressing CD8+ T cell clones are provided for understanding the biology of Chlamydia infection and screening of therapeutics. Furthermore, methods are provided for isolating CD8+ T cells comprising the steps of infecting or identifying a naturally infected mammal with at least one species of Chlamydia causing bacteria, allowing the bacteria to clear or for a T cell immune response to the natural infection, collecting immune splenocytes from the mammal, providing at least one antigen from a Chlamydia-specific bacteria, and expanding and depleting the CD4+ T cell population or purifying the CD8 T cell population to isolate IL-13 expressing CD8+ T cell clones.

### PRIORITY CLAIM

This application is related to and claims the priority benefit of U.S. Provisional Application Ser. No. 61/787,048 to Johnson, filed Mar. 15, 2013. The content of the aforementioned application is hereby incorporated by reference in its entirety into this disclosure.

### STATEMENT REGARDING FEDERALLY SPONSORED RESEARCH

This invention was made with government support under RO1 AI070514 awarded by the National Institutes of Health. The United States Government has certain rights in the invention.

### BACKGROUND

Chlamydia infection is the most common sexually transmitted disease, responsible for about 2.8 million cases a year in the US. 75% of Chlamydia infections effect women. Although 95% of Chlamydia infections can be treated with antibiotics, if left untreated or if complications arise, Chlamydia can lead to uterine scarring, infertility, and potentially fatal ectopic pregnancies.

Fibrosis is a major contributor to scarring and infertility caused by Chlamydia infection, as well as systemic sclerosis (SSc). T cells are the predominant inflammatory infiltrate in affected tissue and are thought to produce cytokines that drive the synthesis of extracellular matrix proteins by fibroblasts, resulting in excess fibrosis. Aberrant interleukin-13 (IL-13) production by peripheral blood effector CD8+ T cells from SSc patients was recently identified to correlate with the extent of skin fibrosis. Although human CD8+ T cells expressing IL-13 were described for the first time in humans, these do not represent a usable tool to study the biology, protein and gene expression in these cells, and screen prospective therapeutics. There is a need for a useful tool to study CD8 T cells that make IL-13 to screen therapeutics to prevent scarring and infertility in Chlamydia-infected females, and progressive scarring in patients with SSc. An isolated CD8+ T cell line expressing IL-13 and methods of isolating CD8+ T cells that make IL-13 is provided.

### BRIEF SUMMARY

In at least one exemplary embodiment of method for isolating Chlamydia-specific CD8+ T cells, the method comprises the steps of recovering immune splenocytes from a mammal previously infected with at least one species of a Chlamydia-causing bacteria; providing the immune splenocytes with at least one antigen to the Chlamydia-causing bacteria; culturing a population of the immune splenocytes under conditions which bring about an increase in the number of CD8+ T cells in the a population and utilizes immune antigen presenting cells; isolating CD8+ T cells from the population; and deriving individual CD8+ T cell clones from the isolated CD8+ T cell, wherein the clones comprise interleukin-13 expressing CD8+ T cell clones. In at least one alternative embodiment, the method may further comprise the steps of infecting a mammal with at least one Chlamydia-causing bacteria or identifying a mammal with a naturally acquired infection; and allowing a period of time to pass sufficient for clearance of the bacteria from the mammal or to allow for a T cell immune response in the mammal with the naturally acquired infection. In at least one alternative embodiment, the method may further comprise the steps of infecting a mammal with at least one Chlamydia-causing bacteria; and allowing a period of time to pass sufficient for clearance of the bacteria from the mammal. The period of time sufficient to allow for a T cell immune response in the mammal with the naturally acquired infection may comprise a period of at least about two weeks.

Furthermore, the step of isolating CD8+ T cells from the population may comprise depleting the CD4+ T cells present in the population. Additionally or alternatively, the step of isolating CD8+ T cells from the population may comprise purifying the CD8+ T cells from the population. In yet another exemplary embodiment of the method of the present disclosure, the immune antigen presenting cells comprise irradiated immune antigen presenting cells. In all embodiments, an immune antigen presenting cell is defined as hematopoietic cells from a mammal that had previously resolved a Chlamydia infection or vaccinated with Chlamydia protein or lipopolysaccharide. The Chlamydia-causing bacteria may comprise a Chlamydia serovar. Additionally or alternatively, the immune splenocytes may comprise immune lymphocytes. Further, in certain embodiments, the mammal of the method may comprise a mouse or a human and the interleukin-13 may comprise murine or human interleukin-13, respectively.

An additional exemplary embodiment of the present disclosure comprises isolated, Chlamydia-specific, interleukin-13 expressing CD8+ T cell clones cultured from a population of immune splenocytes utilizing immune antigen presenting cells. Certain embodiments of the isolated cell clones presented herein may be isolated using a method comprising the steps of: recovering immune splenocytes from a mammal previously infected with at least one species of a Chlamydia-causing bacteria; providing the immune splenocytes with at least one antigen to the Chlamydia-causing bacteria; culturing a population of the immune splenocytes under conditions which bring about an increase in the number of CD8+ T cells in the a population and utilizes the immune antigen presenting cells; isolating CD8+ T cells from the population; and deriving the CD8+ T cell clone from the isolated CD8+ T cells. Furthermore, in at least one embodiment, the immune antigen presenting cells comprise irradiated immune antigen presenting cells.

Additional exemplary embodiments described herein comprise methods for determining a T cell subset comprising the step of identifying a Chlamydia-specific, interleukin-13 expressing CD8+ T cell subset applicable to the diagnosis and/or treatment of one or more disease states. For example, the disease states may comprise scarring associated with a Chlamydia infection and/or systemic sclerosis. Furthermore, the step of identifying a Chlamydia-specific, interleukin-13 expressing CD8+ T cell subset applicable to the diagnosis and/or treatment of one or more disease states of such methods may comprise employing a gene expression microarray or a proteomics-based methodology such as MALDI-TOF mass spectroscopy.

### BRIEF DESCRIPTION OF THE DRAWINGS

The present invention will be better understood and features, aspects and advantages other than those set forth above will become apparent in light of the following detailed description thereof. Such detailed description makes reference to the following drawings, wherein:

FIG. 1 shows Chlamydia-specific CD8 T cell clones. A) CD8 T cells are a minor subset in bulk Chlamydia-specific T cell populations expanded ex vivo using either UV-inactivated C. muridarum (uvMoPn) or soluble antigen. Soluble antigen was more effective than uvMoPn for CD8 T cell expansion. B) Specificity of five CD8 T cell clones derived using irradiated immune splenocytes as feeder APC. Even with irradiation, immune splenocytes make cytokines that can support non-specific T cell proliferation. Irradiated naive splenocytes do not support non-specific T cell proliferation. CD8bm1 is an alloreactive CD8 T cell clone specific for H-2K^{bm1}; and

FIG. 2 shows mapping of MHC restriction elements: A) Shows that naive irradiated splenocytes exposed to Chlamydia antigen (uvMoPn) do not activate bystander CD8 T cells, in this case CD8bm1, an alloreactive CD8 T cell clone specific for H-2K^{bm1}. On that basis irradiated naive splenocytes can be used as antigen presenting cells for testing T cell clone recognition of Chlamydia antigens. Conversely, irradiated immune splenocytes cannot be used for this purpose because their activation by exposure to uvMoPn leads to activation of the bystander CD8 T cell clone CD8bm1. B) shows that the majority of the CD8 T cell clones were not dependent on MHC class Ia molecules for recognition of Chlamydia antigens, matching the Chlamydia CD8 T cell clone data from humans. Specifically, CD8 T cell clones sAg-1, sAg-2, sAg-3 recognized irradiated naive splenocytes from wild type (C57) and K^{b} D^{b} dual knockout mice lacking MHC class 1a molecules (K^{b}D^{b}) equally well when the respective irradiated splenocytes were exposed to uvMoPn; and

FIG. 3 shows cytokine patterns in CD8 T cell clones. The CD8 T cell clones were activated for 24 h with immobilized anti-CD3 and their cytokine patterns determined by ELISA. All were capable of making IL-2, all produced large amounts of IFN-γ and TNF-α, most produced large amounts of IL-10, and two produced large amounts of IL-13 (sAg-1 and sAg-3); and

FIG. 4 shows CD8 T cell clones ability to terminate C. muridarum replication in upper reproductive tract epithelial cells. C57epi.1 cells untreated (A) or pretreated with 10 ng IFN-γ (B) prior to infection. T cells were added to infected epithelial cells 4 h post-infection at an E:T ration of 0.75:1; wells harvested 32 h post-infection and recovered IFU quantified on McCoy monolayers. Top panels experiment #1; bottom panels experiment #2; and

FIG. 5 shows expression of perforin and Plac8 in CD8 T cell clones. A) All the CD8 clones express levels of perforin comparable to a conventional alloreactive CD8 T cell clone designated CD8bm1, and do not express Plac8. B) Positive control for the Plac8 PCR reagents using total RNA from previously published CD4 T cell clones that do (uvmo-2) and do not (uvmo-4) express Plac8; and

FIG. 6 shows that T cell culture methodologies based on irradiated naive antigen-presenting cells (splenocytes) pulsed with UV-inactivated Chlamydia select out only CD4 T cells in vitro.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments above and claims below for interpreting the scope of the invention.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation in scope of this disclosure is thereby intended. Indeed, the materials, systems, and methods of the present disclosure may be embodied in many different forms and should not be construed as being limited to the embodiments expressly set forth herein.

Likewise, many modifications and other embodiments of the materials, systems and methods set forth herein will come to mind to one of skill in the relevant arts having the benefit of teachings presented herein. Therefore, it is to be understood that any such alterations, modifications, embodiments and further applications of the principles of the present disclosure are intended to be included within the scope of the appended claims. Although specific terms may be employed herein, they are used in a generic and descriptive sense only and not for purposes of limitation.

Unless defined otherwise, all technical and scientific terms used herein have the same meaning as commonly understood by one of skill in the relevant arts. Although any methods and materials similar to or equivalent to those described herein can be used in the practice or testing of the present invention, the preferred methods and materials are described herein.

The disclosure of the present application provides for isolated Chlamydia-specific CD8+ T cells that express interleukin-13 (IL-13) and various methods of isolating and using the same. Specifically, certain embodiments of the CD8+ T cells and methods disclosed herein provide useful insights into Clamydia-associated immunopathology.

In general, CD8 T cell subsets have a role in protection and immunopathology during Chlamydia genital tract infections. As described in more detail below, the Chlamydia-specific CD8 T cells clones described herein are a minority subset in polyclonal T cell populations that were expanded in vitro from C57BL/6 mice that previously cleared C. muridarum genital tract infections. Because CD8 T cell response has been associated with immunopathology, it is important to understand the nature of Chlamydia-specific CD8 T cell responses. Cloning Chlamydia-specific CD8 T cells from polyclonal populations derived from immune mice reveals that the majority of CD8 T cells are not restricted by MHC class Ia molecules and have cytokine production patterns that are consistent with immunopathology (methods and materials described in further detail in Johnson et. al, 2014, Immunology Jan 15. doi: 10.1111/imm.12248. PMID: 24428415 (available at http://www.ncbi.nlm.nih.gov/pubmed/24428415) (accessed Mar. 16, 2014), which is hereby incorporated herein by reference in its entirety). While cytokine patterns provide a link to immunopathology, some of these CD8 T cell clones are able to control C. muridarum replication in reproductive tract epithelial cells in vitro. Accordingly, all mechanisms of clearance are not equal. Information gleaned from these novel CD8 T cell clones correlates with the CD8 T cells' role in immunopathogenesis in vivo.

Because Chlamydia species are intracellular pathogens, early efforts to understand protective immunity focused on CD8 T cells, including isolation and characterization of CD8 T cell clones that were protective against infection (Igiesteme et. al, 1994, Infect Immun 62:5195-5197). However the importance of CD8 T cells to protective immunity was brought into question by depletion studies and knockout mice. Indeed, it was shown that CD8-depleted and b2 microglobulin knockout mice were not compromised in their ability to clear C. muridarum genital tract infections (Morrison et. al. 1995, Infect Immun 63:4661-4668). In parallel, human studies with Chlamydia-specific CD8 T cell clones isolated from infected individuals showed that the majority of the T cells clones were not restricted by classical or non-classical MHC class I molecules (Gervassi et. al, 2003, J Immunol 171:4278-4286 and Matyszak et. al, 2004, Infect Immun 72:4357-4367).

Recent studies in the C. muridarum mouse model have implicated CD8 T cells in immunopathology and infertility (Igietseme et al., 2009, J Infect Dis 200:926-934). Indeed, research has shown that immune responses mediated by CD8 T cells are major contributors to the pathology of Chlamydia. Additionally, tumor necrosis factor alpha (TNF-α) was found to be critical for CD8 T cell mediated immunopathology (Murthy et al., 2011, Infect Immun 79:2928-2935).

The identity of the CD8 T cell subset(s) mediating immunopathology during genital tract infections is not well understood in mouse or man. To address this issue Chlamydia-specific CD8 T cell clones were isolated from mice that had previously cleared C. muridarum genital tract infections and investigated their immunobiology including restriction elements, cytokine patterns and the ability to terminate Chlamydia replication in upper reproductive tract epithelial cells.

To better understand Chlamydia pathogenesis, the Chlamydia-specific T cell clones were derived from immune mice using UV-inactivated-C.muridarum-pulsed naïve splenocytes as antigen presenting cells (APC) (Jayarapu et al. 2009, Infect Immuno 77:4469-4479). Under those conditions, polyclonal T cell cultures were about 100% CD4 T cells (see FIG. 6). Isolating Chlamydia-specific CD8 T cells clones from mice was performed in order to study their immunobiology and compare them with Chlamydia-specific CD8 T cells that have been described in humans.

Specifically, 4-5 week old female C57BL/6 mice from Harlan Labs (Indianapolis, Ind.) and K^{b}D^{b} double knockout female mice (lack MHC class Ia molecules) from Taconic (Hudson, N.Y.) were used. C57epi.1 epithelial cells and McCoy fibroblasts were cultured. C57BL/6 female mice were infected vaginally with C. muridarum (Nigg). After bacterial clearance, CD8 T cell clones were derived from immune splenocytes after expansion on irraditated immune splenocytes and Chlamydia antigen, and depletion of CD4 T cells, by limiting dilutions using either UV-inactivated C. muridarum (mycoplasma-free Chlamydia muridarum (Nigg) (MoPn)) or elementary body (EB)-depleted soluble C. muridarum antigen preparations as the stimulus (about 99.998% depletion of EB by centrifugation). (Johnson et. al, 2014, Immunology Jan 15. doi: 10.1111/imm.12248. PMID: 24428415).

While in the foregoing embodiment the mice were artificially infected with the bacteria, a mammal with a naturally acquired infection could alternatively be used to derive the CD8 T cell clones. In such case, the CD8 T cell clones can be derived pursuant to the methods described herein after the infection has been given a sufficient time to allow for a T cell immune response in the infected mammal (typically longer than about two weeks).

An alternative culture system based on immune Ig-receptor-bearing APC may utilize cross-presentation pathways to generate more CD8 T cell epitopes and promote greater expansion of CD8 T cells. Activating lymphocytes from immune mice with immune-irradiated splenocytes pulsed with either UV-C. muridarum (uvMoPn) or the EB-depleted-infected-epithelial-cell lysates (soluble Chlamydia antigens (sAg)) yielded polyclonal T cell populations with small, but readily detectable, CD8 T cell populations (about 3-10%) (see FIG. 1). Immune-irradiated splenocytes pulsed with UV-inactivated-C.muridarum expanded a small but detectable CD8 T cell population (−3%), while those immune-irradiated splenocytes pulsed with sAg expanded a bit larger of a detectable CD8 T cell population (−10%) (FIG. 1). CD4 T cells were selectively depleted from those polyclonal populations using magnetic bead technology and the remaining T cell populations were cloned by limiting dilution to derive two “UV-C. muridarum” CD8 T cell clones (labeled in the Figures as “8uvmo-1” and “8uvmo-2”) and three “soluable antigen” CD8 T cell clones (which are labeled in the Figures as “sAg-1,” “sAg-2” and “sAg-3”). Accordingly, CD8 clones from 2 mice were isolated by limiting dilution after CD4 T cell depletion from polyclonal populations by magnetic bead separation (see FIG. 1b).

As shown herein, conventional MHC class Ia—restricted Chlamydia-specific CD8 T cells do not appear to be the dominant CD8 T cell type in Chlamydia-specific T cell populations expanded from immune mice. Indeed, the majority of the Chlamydia-specific CD8 T cell clones are not restricted by MHC class Ia molecules (see FIG. 2 and, in particular, FIG. 2b). Specifically, 3 of the C57BL/6-derived CD8 T cell clones were activated as well or better by MHC class Ia deficient naïve splenocytes pulsed with uvMoPn as compared to those derived from syngeniec naïve C57BL/6 pulsed with uvMoPn. Furthermore, two atypical Chlamydia-specific CD8 T cells have an unusual cytokine polarization that includes combinations of IFN-γ, TNF-α, IL-10, and IL-13 (FIG. 3) representing the successful application of the methodology described herein. . Here, the CD8 T cell clones had varying abilities to terminate Chlamydia replication in epithelial cells (see FIGS. 4). 8uvmo-2 and 8uvmo-3 were effective in terminating C. muridarum replication; however, 8sAg-1, 8sAg-2, and 8sAg-3 were unable or inefficient at terminating replication even when the epithelial monolayers were pretreated with IFN-γ. Additionally, all the CD8 T cell clones expressed perforin and none had detectable Plac8 mRNA (see FIG. 5). Finally and summarily, as illustrated in FIG. 3, two of the five CD8 T cell clones produced large amounts of IL-13 in addition to IL-2, TNF-α, IL-10, and IFN-γ.

IL-13 producing Chlamydia-specific CD8 T cells may contribute to immunopathology during C. muridarum genital tract infections based on the roles of TNF-α and IL-13 in scar formation. Specifically, data shows that TNF-α is associated with immunopathology and IL-13 is detrimental to Chlamydia clearance and associated with fibrosis and residual scarring. CD8 T cells producing IL-10, IL-13 and TNF-αare interesting with respect to immunopathology because in addition to a role for IL-10 in scarring, the combination of TNF-α and IL-13 is the underlying mechanism for bleomycin-induced pulmonary fibrosis and TNBS-induced colonic fibrosis in mouse models. Further, IL-13 expressing CD8 T cells may mediate immunopathology in systemic sclerosis, a rheumatologic disorder that manifests as progressive scarring of the skin, and Clamydia-specific CD8 T cells making IL-10, IL-13 and TNF-α may contribute to the CD8-mediated immunopathology observed in experimental murine genital tract infections. Accordingly, the atypical CD8 T cell clones described herein are an important effector T cell subset for Chlamydia-associated immunopathology and a representative of the atypical CD8 T cells previously described in humans.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the present disclosure was illustrated using specific examples, theoretical arguments, accounts and illustrations, these illustrations and the accompanying discussion should by no means be interpreted as limiting the technology.

1. A method for isolating Chlamydia-specific CD8+ T cells comprising the steps of:
recovering immune splenocytes from a mammal previously infected with at least one species of a Chlamydia-causing bacteria;
providing the immune splenocytes with at least one antigen to the Chlamydia-causing bacteria;
culturing a population of the immune splenocytes under conditions which bring about an increase in the number of CD8+ T cells in the a population and utilizes immune antigen presenting cells;
isolating CD8+ T cells from the population; and
deriving individual CD8+ T cell clones from the isolated CD8+ T cells, wherein the clones comprise interleukin-13 expressing CD8+ T cell clones.

2. The method of claim 1, wherein the Chlamydia-causing bacteria comprises a Chlamydia serovar.

3. The method of claim 1, wherein the immune splenocytes comprise immune lymphocytes.

4. The method of claim 1, further comprising the steps of:
infecting a mammal with at least one Chlamydia-causing bacteria or identifying a mammal with a naturally acquired infection; and
allowing a period of time to pass sufficient for clearance of the bacteria from the infected mammal or to allow for a T cell immune response in the mammal with the naturally acquired infection.

5. The method of claim 1, wherein the step of isolating CD8+ T cells from the population comprises depleting the CD4+ T cells present in the population.

6. The method of claim 1, wherein the step of isolating CD8+ T cell from the population comprises purifying the CD8+ T cells from the population.

7. The method of claim 1, wherein the mammal comprises a mouse or a human and the interleukin-13 comprises murine or human interleukin-13, respectively.

8. The method of claim 1, wherein the CD8+ T cell clones do not express Plac8.

9. The method of claim 1, wherein the immune antigen presenting cells comprise irradiated immune antigen presenting cells.

10. The method of claim 4, wherein the period of time sufficient to allow for a T cell immune response in the mammal with the naturally acquired infection comprises at least about two weeks.

11. An isolated, Chlamydia-specific, interleukin-13 expressing CD8+ T cell clone cultured from a population of immune splenocytes utilizing immune antigen presenting cells.

12. The isolated cell clone of claim 11, wherein the cell clone does not express Plac8.

13. The isolated cell clone of claim 12, isolated using a method comprising the steps of:
recovering immune splenocytes from a mammal previously infected with at least one species of a Chlamydia-causing bacteria;
providing the immune splenocytes with at least one antigen to the Chlamydia-causing bacteria;
culturing the population of the immune splenocytes under conditions which bring about an increase in the number of CD8+ T cells in the a population and utilizes the immune antigen presenting cells;
isolating CD8+ T cells from the population; and
deriving the CD8+ T cell clone from the isolated CD8+ T cells.

14. The isolated cell clone of claim 13, wherein the immune antigen presenting cells comprise irradiated immune antigen presenting cells.

15. A method for determining a T cell subset comprising the step of identifying a Chlamydia-specific, interleukin-13 expressing CD8+ T cell subset applicable to the diagnosis and/or treatment of one or more disease states.

16. The method of claim 15, wherein at least one of the disease states comprises scarring associated with a Chlamydia infection.

17. The method of claim 15, wherein at least one of the disease states comprises systemic sclerosis.

18. The method of claim 15, wherein the step of identifying a Chlamydia-specific, interleukin-13 expressing CD8+ T cell subset applicable to the diagnosis and/or treatment of one or more disease states comprises employing a gene expression microarray.

19. The method of claim 15, wherein the step of identifying a Chlamydia-specific, interleukin-13 expressing CD8+ T cell subset applicable to the diagnosis and/or treatment of one or more disease states comprises employing a proteomics-based methodology.

20. The method of claim 19, wherein the proteomics-based methodology comprises MALDI-TOF mass spectroscopy.
